(19) United States
(12) Patent Application Publication
(10) Pub. No.: US 2019/0250088 A1
(43) Pub. Date: Aug. 15, 2019

(54) Methods and Apparatus for Predicting and Confirming Drug-Induced Thrombocytopenia Through Particle Detection with Dynamic Light Scattering

(71) Applicant: LightIntegra Technology Inc., Vancover (CA)

(72) Inventors: Elisabeth Maurer, Vancouver (CA); Audrey Labrie, Vancouver (CA); Jennnifer Chiang, Vancouver (CA)

(73) Assignee: LightIntegra Technology Inc., Vancouver (CA)

(21) Appl. No.: 16/269,668

(22) Filed: Feb. 7, 2019

Related U.S. Application Data
(60) Provisional application No. 62/630,858, filed on Feb. 15, 2018.
(60) Provisional application No. 62/720,055, filed on Aug. 20, 2018.

(51) Int. Cl. G01N 15/02; G01N 33/86

(52) U.S. Cl. CPC G01N 15/0211; G01N 2015/0222; G01N 33/86

(57) ABSTRACT

Dynamic light scattering (DLS)-based particle testing is used for screening to predict drug-induced complications with heparin and other compounds known to lead to thrombocytopenia in many patients. The measurement of particle content is used to both predict as well as confirm drug-induced thrombocytopenia (“DIT”).

### TECHNICAL FIELD

This invention relates to Drug-Induced Thrombocytopenia (DIT) and more specifically to apparatus and methods for reliably predicting and confirming DIT through particle detection using dynamic light scattering techniques.

### BACKGROUND

Thrombocytopenia is the technical term for low platelet count which is a characteristic side effect of treatment with certain drugs. Heparin-induced thrombocytopenia (HIT) is one example of drug-induced thrombocytopenia (DIT). HIT is a transient, immune-mediated adverse drug reaction in patients recently exposed to heparin that generally produces thrombocytopenia and often results in venous and/or arterial thrombosis. Unlike other forms of thrombocytopenia, HIT is generally not marked by bleeding. HIT occurs in up to 5% of patients receiving unfractionated heparin (UFH) and in <1% who receive low molecular weight heparin (LMWH). HIT is characterized by immunoglobulin G (IgG) antibodies that recognize an antigen complex of platelet factor 4 (PF4) bound to heparin. These pathologic antibodies trigger a highly prothrombotic state by causing intravascular platelet aggregation, intense platelet, monocyte and endothelial cell activation and excessive thrombin generation. Microparticles are released from these activated platelets and contribute to the thrombotic effect of HIT.

### Clinical Features

HIT typically presents with a fall in platelet count with or without venous and/or arterial thrombosis.

Thrombocytopenia: A platelet count fall >30% beginning 5-10 days after heparin exposure, in the absence of other causes of thrombocytopenia, might be HIT, unless proven otherwise. A more rapid onset of platelet count fall (often within 24 hours of heparin exposure) can occur when there is a history of heparin exposure within the preceding 3 months. Bleeding is very infrequent.

Thrombosis: HIT is associated with a high risk (30-50%) of new venous or arterial thromboembolism. Thrombosis may be the presenting clinical manifestation of HIT or can occur during or shortly after the thrombocytopenia.

Other clinical manifestations of HIT: Less frequent manifestations include heparin-induced skin lesions, adrenal hemorrhagic infarction, transient global amnesia, and acute systemic reactions (e.g. chills, dyspnea, cardiac or respiratory arrest following IV heparin bolus).

### Health Economic Impact

Over 1 trillion units of the anticoagulant heparin are used in more than 12 million patients per year for treating and preventing thromboembolic disorders in medical and surgical patients. These 12 million patients make up one third of hospitalized patients per year in the United States. With a mode prevalence rate of 2.75%, 330 000 patients per year have HIT; this can range from 60,000 to 600,000 patients per year in the US. Based on the average cost of treatment per case of HIT, this costs from $863 Million to $8.6 Billion annually. This is extrapolated from a study by Smythe et al. for the US National Library of Medicine (2008). On average, HIT case patients incurred a financial loss of $14,387 per patient and an increase in length of stay of 14.5 days.

Several clinical studies in western countries have confirmed that the prevalence of HIT is ˜0.5-5% depending on the clinical setting (UFH therapy is on the higher side of this range while LMWH therapy is on the lower end). It has been reported that of these cases, half experience complications and roughly 90,000 die.

Earlier diagnosis and stopping heparin administration sooner before complications arise would be key to reducing the treatment cost.

### Current Diagnostic Methods for HIT as an Example

The diagnosis of HIT is based on both clinical and serological findings. Current testing methods are technically complex, slow to perform and sometimes take days to return results.

Clinically, HIT is diagnosed by the 4T test. It is a positive scale that rates symptoms from 0 to 2 with a higher score indicating more extreme symptoms. The scores from the individual symptoms are then summed to give the total score. The test is ˜100% sensitive due to the nature of the criteria for scoring a 0 in each section.

Currently the 4T scale is used to clinically give a score for the risk a person developed HIT.

TABLE 1
4T test scoring

| Category | 2 points | 1 point | 0 point |
|---|---|---|---|
| Thrombocytopenia | >50% fail, or nadir | 30-50% fail, or | <30% fail, or nadir. |
| | ≥20 × 10⁹/L | nadir 10-19 × 10⁹/L | <10 × 10⁹/L |
| Timing of the | Days 5 to 10, or | >Day 10 or timing | < Day 4 |
| decrease in | <day 1 with recent | unclear or <day 1 | (no recent heparin) |
| platelet count | heparin (past 30 | if heparin exposure | |
| | days) | within past 30-100 | |
| | | days | |
| Thrombosis or | Proven thrombosis, | Progressive, | None |
| other sequelae | skin necrosis, or | recurrent, or silent | |
| | acute systemic | thrombosis, | |
| | reaction after | erythematous skin | |
| | heparin bolus | lesions | |
| Other causes of | None evident | Possible | Definite |
| thrombocytopenia | | | |

The higher the 4T score the higher the chance that the patient has HIT. Studies have shown that only 50% of the high-risk patients, as given by the 4T scale, are confirmed by laboratory HIT testing. Because the current confirmatory laboratory tests are slow and technically demanding, physicians might opt against ordering them as it could be argued that the cost outweighs the benefit when only 50% of cases will receive a delayed confirmation for HIT.

### Currently Used Confirmatory Laboratory Tests

The main method is a Solid-Phase Immunoassay for anti-platelet factor 4 antibody (anti-PF4 ELISA). The antibodies are detected by reaction with surface-bound PF4/heparin. These immunoassays are technically easy to perform and usually take somewhere in the range of 0.5-4 hours. While these immunoassays are inexpensive, commercially available and exhibit a high sensitivity for HIT II antibodies (in the range of 80-100%), they have low specificity which leads to a high rate of false positive results. Following a positive ELISA result confirmation with a functional test is still required.

The two main functional tests to confirm HIT are the ¹⁴C-Serotonin Release Assay (SRA) and a Heparin-Induced Platelet activation test (HIPA). Of the two functional tests, the SRA takes much longer to perform, and because of the use of radiolabeled serotonin can only be performed in special laboratories. Platelets from pedigree donors are loaded with ¹⁴C-Serotonin and the percentage of serotonin released is measured after addition of patient serum and heparin.

HIPA is a platelet-activation test in which the patient's serum is mixed with pedigree donor platelets in the presence of heparin. Aggregation of the donor platelets indicates the presence of antibodies to the heparin-PF4 complex. The HIPA test also requires special equipment and trained users. The HIPA test is easier to perform than the SRA but far less accurate (35%-85%).

Recently, increased expression of CD62 on the surface of platelets in response to patient serum and heparin has also been explored as a confirmatory test using flow cytometry. As an example, U.S. Pat. No. 9,851,367 describes a method of detecting platelet activation comprising the steps of a) obtaining a blood sample from a patient suspected of having heparin-induced thrombocytopenia (HIT); b) incubating an effective amount of platelet factor 4 (PF4) with a sample of platelets to yield a sample of PF4-treated platelets; c) contacting the patient blood sample with the PF4-treated platelets; and d) measuring the extent of platelet activation, wherein an increase in platelet activation compared with results obtained using a normal blood sample is indicative of the patient having HIT.

No single assay has high sensitivity and specificity. Using ELISA and functional testing in combination is the most accurate but also the most time-consuming method.

### SUMMARY OF THE INVENTION

In view of the foregoing shortcomings with existing protocols there is a need for a predictive test as well as a more rapid, less sophisticated confirmatory test for DIT.

### BRIEF DESCRIPTION OF THE DRAWINGS

Further features and advantages of the present invention will become apparent from the following detailed description, taken in combination with the appended drawing, in which:

FIG. 1 is a graph illustrating the scattering intensity of all particles in a sample as analyzed with dynamic light scattering.

FIG. 2 is a schematic overview of test sample preparation for the confirmatory test for HIT as performed in the SRA functional test.

FIG. 3 is a graph illustrating the prediction of SRA-positivity by measurement with dynamic light scattering of heat-inactivated patient serum.

FIG. 4 is a schematic overview of the confirmatory functional test according to the present invention resulting from dynamic light scattering analysis.

FIG. 5 is a graph showing the effect of heat-inactivation on patient serum.

FIG. 6 is illustrates the calculation of reaction mixture signal.

FIGS. 7 and 8 are a comparison of reaction change, specifically, illustrating the effect of activated compared to non-activated platelets in the pedigree donor platelet sample (PRP).

FIG. 7 illustrates reaction change for low positive samples with platelets from a fresh activated pool; and

FIG. 8 illustrates reaction change for a fresh non-activated donor.

FIG. 9 is a tabular overview of the results of test results based on the preliminary protocol for sample preparation.

### DETAILED DISCLOSURE OF PREFERRED EMBODIMENTS

### Definitions Used Herein

DIT—Drug Induced Thrombocytopenia

HIT—Heparin Induced Thrombocytopenia

LMWH—Low Molecular Weight Heparin

UFH—Unfractionated heparin

SRA—¹⁴C-Seratonin Release Assay

HIPA—Heparin-Induced Platelet activation test

IC—immune complexes

DLS—Dynamic Light Scattering

PRP—platelet rich plasma

PFP—platelet free plasma

% MP—microparticle content: The percent of the total intensity of the size distribution in the Gated MP range (between 50 nm and 550 nm)

TLX—a commercially available instrument sold under the trademark ThromboLUX, manufactured by the applicant and assignee for the present application. These instruments analyze samples using dynamic light scattering (herein, as noted, “DLS”) methodologies and instruments as described in U.S. Pat. Nos. 7,341,873 and 8,877,458; the entire disclosures of which are incorporated herein by this reference and each of which is licensed by the assignee of the present application.

MP—microparticles. The term “microparticles” as used herein is understood to mean particles within bodily fluids (such as blood), which have a hydrodynamic radius of less than about 1 micron, and may in one possible embodiment have a hydrodynamic radius of between approximately 20 and 550 nm, and more preferably in another embodiment may have a hydrodynamic radius of between about 50 nm and 499 nm. The term microparticles as used herein is also intended to include so-called “nano-particles”. Microparticles are much smaller than the larger platelets in a platelet rich plasma blood sample for example. This may be seen in exemplary differential interference contrast (DIC) microscopy images of platelet rich plasma samples taken from a cardiovascular disease patient, showing the presence of microparticles in the fluid along with the bigger platelets.

Particles. As used herein, “particles” refers to all DLS-signal producing elements of a patient sample, including platelets, microparticles and aggregates.

Patient Sample collectively comprises patient PRP, plasma and/or patient serum.

Microparticles indicate platelet activation. As described in detail in, for instance, U.S. Pat. Nos. 8,323,922 and 8,834,129 (“Dynamic Light Scattering for in vitro Testing of Bodily Fluids”), the entire disclosures of which are incorporated herein by this reference and which are licensed by the assignee of the present application, dynamic light scattering methodologies are useful to detect microparticles as an indicator of platelet activation status.

DLS-based particle testing may be used for pre-screening to predict drug-induced complications with heparin being an example for a drug known to lead to thrombocytopenia in many patients. However, many other pharmaceuticals such as cefepime etc. have been shown to cause thrombocytopenia.

The measurement of particle content may be used to both predict as well as confirm drug-induced thrombocytopenia. To predict the risk of a patient for DIT, blood particle content is measured before the patient receives a DIT-inducing drug such as heparin and the patient platelets could be stimulated with the drug such as heparin in vitro to determine whether the patient's platelets are responsive to the drug. To confirm DIT once a reaction has been observed in vivo, the patient's serum and the implicated drug can be added to pedigree donor platelets to replicate the reaction in vitro and confirm that the drug is causing a problem in the patient.

### Current Treatment

Existing protocols outline the following steps as the first things to do in a suspected case of HIT, according to the “4T” clinical test.

Immediate cessation of all formulations of heparin is mandatory including heparin flushes, heparin coated catheters, heparinised dialysate and any other sources

Send blood samples for laboratory confirmation

Initiate alternative anticoagulation. The duration of treatment is not well defined; however, it should be continued for at least 2-3 months to prevent recurrence of thrombosis

Monitor carefully for thrombotic event

Monitor platelet count till recovery

Warfarin should not be used until the platelet count has recovered

Avoid prophylactic platelet transfusion because they may exacerbate the hypercoagulable state, leading to additional thrombosis; however, if the patient develops bleeding or is undergoing major surgical intervention, therapeutic platelet transfusion can be considered.

The indirect factor Xa inhibitor fondaparinux (Arixtra) is not approved for use in HIT, but some experts consider it an important treatment option, especially in stable, non-critically ill patients. Several novel oral anticoagulants exist: rivaroxaban, dabigatran, and apixaban and preliminary evidence suggests that they may be beneficial for HIT, particularly in cases refractory to standard therapies. However, these agents have not been fully assessed for treatment of patients with HIT and none have FDA approval for use in HIT. Patients might benefit from the predictive test described below before being treated with these drugs.

### Predicitve and Confirmatory Tests for DIT According to the Invention Predictive Test

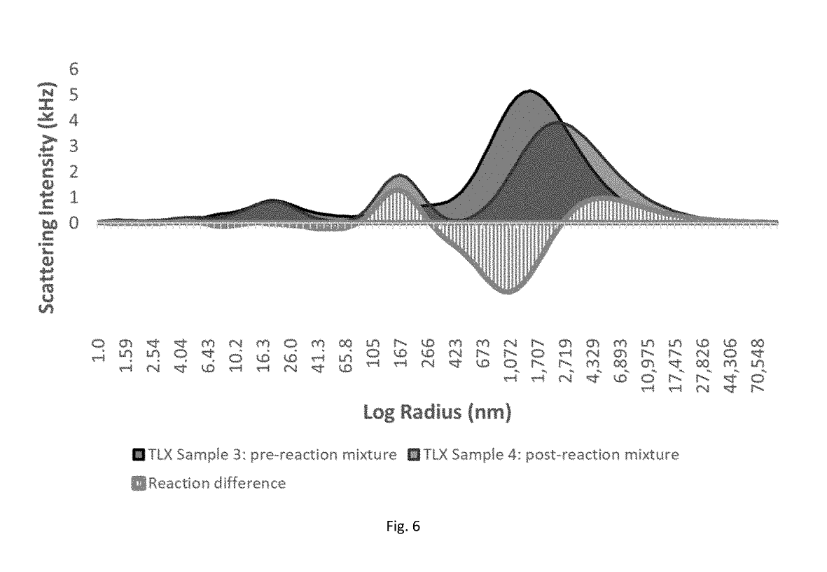

Prior experimental research has relied upon gated flow cytometry optimized for Microparticle detection to evaluate the risks of DIT—using gated flow cytometry the assay necessarily ignores signals from other components in the patient sample, including platelets and small aggregates. In contrast to this approach the present invention uses DLS to evaluate all particles in the patient sample, including platelets, aggregates and microparticles. This approach mimics more closely a thrombocytopenic event as it might occur in vivo and is a more accurate prediction of risk to the patient. The approach adopted herein is analogous to a combination of a microparticle-only test with a HIPA test. Particle content redistribution in the reaction mixture to obtain reaction difference according to the invention is discussed below in respect of FIGS. 6, 7 and 8.

DLS determines all particles suspended in a patient or donor sample by analyzing their speed of Brownian motion. Microparticles, platelets and small aggregates are detected simultaneously. Particles of different sizes give rise to different DLS signals and the total of all signals together (total intensity measured in kHz) is an indicator of overall particle concentration in the sample. Platelet activation can cause microparticle generation (a peak appears or increases in the range of radii below about 500 nm), changes in platelet size (shift in mean radius) and/or platelet aggregation (shift of the platelet peak to larger radii or, when aggregates settle out, a reduction in total intensity). Because platelet activation in response to DIT-causing drugs can result in a multitude of combinations of all these different effects, it is important to measure the entire particle composition of the sample with DLS before and after addition of the drug to accurately determine the risk of DIT; the inventions herein are based on the measurement of the entire particle composition. The DLS methodology is therefore substantially different from microparticle-gated flow cytometry (detecting only microparticles but not platelets or platelet aggregates) or platelet aggregation assays like the HIPA (detecting only platelet aggregation and not microparticles).

Another possibility to determine an indicator of risk for DIT utilizes the difference of the DLS results between the calculated mixture (particle size distribution predicted based on the components that are mixed together) and the actual mixture. Said another way, the reaction might be occurring very rapidly—perhaps faster than can actually be measured—resulting in a difference between the sum of the components and the actual mixture that intensifies over time. Thus, the difference between the calculated and the actual mixture may show that the reaction has occurred and therefore eliminate the need to perform a confirmatory test.

The predictive test according to the present invention involves comparing the size distribution test results of patient platelets before and after addition of the drug in question, for example heparin.

Drug-induced thrombocytopenia (DIT) may be predicted by

determination of the formation of immune complexes in patient serum in response to addition of the drug to be tested and observation of changes in the particle size distribution measured with dynamic light scattering or an equivalent technology that is sensitive to sub-micron particles even in the presence of micron-sized particles; the activation response is detected by a change in size distribution, specifically a change in relative content of particles smaller than 550 nm radius (microparticles) and a change in relative content of particles larger than 550 nm radius;

the activation response of patient platelets following the addition of the drug in question.

The predictive test may be broadly characterized as follows:

A method of detecting the formation of immune complexes in platelet rich plasma in order to predict the risk of drug-induced thrombocytopenia (DIT), the method comprising the steps of:
a. obtaining a sample of platelet rich plasma from a patient;
b. adding to the sample a compound that is suspected to cause a reaction with the platelets;
c. analyzing the sample using dynamic light scattering (DLS) to determine platelet activation in the sample;
d. comparing the determined platelet activation to predetermined thresholds.

For the predictive test described herein the PRP sample from the patient would still contain enough platelets that the reaction with the DIT-inducing drug may be tested in vitro. After the reaction has occurred in the patient the patient platelets are mostly gone—due to thrombocytopenia. Hence, a confirmatory test as described below.

### Confirmatory Test

Drug-induced thrombocytopenia (DIT) may be confirmed by:

the activation response of platelets from a donor—identified as a pedigree donor by the microparticle content in their platelet-rich plasma—following the addition of patient serum and the drug in question; in the example of heparin-induced thrombocytopenia, immune complexes are formed between the pathological antibody and the complex of heparin, the drug, and platelet factor 4 (PF4), released from patient platelets. The immune complexes activate donor platelets which can be measured as significant change in the size distribution; drug-induced thrombocytopenia is therefore confirmed by the measurement of changes in sample composition when platelet-rich plasma of a pedigree donor is combined with a patient serum in the presence of a therapeutic dose of the drug.

If the reaction is negative the absence of the pathologic antibody in the patient serum can be confirmed by adding a commercial antibody known to elicit a HIT-like reaction through the formation of immune complexes resulting in a positive reaction.

If the reaction is positive an inhibitor is added to prevent the positive reaction for example by adding a 100 times higher heparin concentration to the mixture of donor platelets and patient serum.

Heparin is only an example of a drug that can lead to drug-induced thrombocytopenia and there are many other drugs that are known to lead to the condition, including for example, the compounds listed below in Table 2. Herein, drugs that can lead to drug-induced thrombocytopenia such as but not limited to those in Table 2 are collectively referred to as “DIT inducing compounds.”

TABLE 2

| Type | Examples |
|---|---|
| Hapten- | Penicillin and penicillin derivatives |
| induced | |
| antibody | |
| Drug- | Quinidine, quinine, NSAIDs, various |
| dependent | antibiotics, sedatives, anticonvulsants |
| antibody | |
| Ligand mimetic | Tirofiban, eptifibatide, roxifiban |
| Drug-specific | Abciximab |
| antibody | |
| Drug-induced | Gold salts, procaine amide |
| autoantibody | |
| Immune | heparin |
| complex | |

The confirmatory test may be broadly characterized as follows:

A method of detecting the formation of immune complexes in platelet rich plasma in order to confirm drug-induced thrombocytopenia (DIT), the method comprising the steps of:
obtaining a first sample of platelet rich plasma, plasma or serum from a patient;
obtaining a second sample of platelet rich plasma from a pedigree donor;
adding to the first sample a compound that is suspected to cause a reaction with platelets;
combining the second sample of platelet rich plasma with the combination of the first sample and the compound;
analyzing the combination formed in step [0076] before and after a reaction time using dynamic light scattering (DLS) to determine platelet activation;
comparing the determined platelet activation to predetermined thresholds.

### Limitations

The reported diagnostic limitations of other tests of heparin-induced thrombocytopenia need to be considered when comparing results from these tests with the results obtained with the DLS test. The DLS test might be limited if patient sera already contain high concentrations of microparticles or the qualification of sensitive donors is difficult.

### Methodology

### Overview

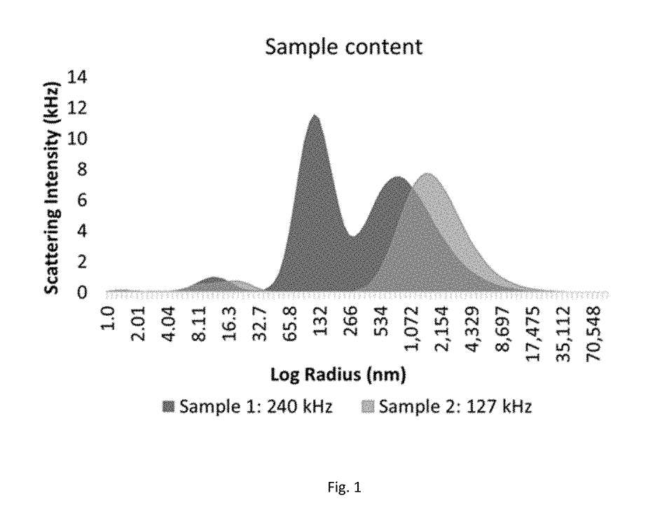

ThromboLUX uses the principle of Dynamic Light Scattering—also known as photon correlation spectroscopy or quasi-elastic light scattering—to measure the composition of samples based on the relative amounts of differently-sized particles such as microparticles and platelets in a sample. These measurements are performed by illuminating the suspended particles with laser light and then analyzing the time variation of the scattered light's intensity. This analysis provides information on the size, relative concentration and distribution of the sample components. Using ThromboLUX, the scattering intensity of particles and their mean radius can be quantified. The scattering intensity of all particles in a sample is given as the area under the curve of the sample histogram FIG. 1. The presence of different microparticle populations indicates product heterogeneity - peaks with mean radii of about 15 nm and 120 nm in FIG. 1.

Sample content is visualized by the histogram area and quantified by the overall scattering intensity in kHz. A high number of microparticles in a sample of activated platelets (Sample 1) contribute a high level of scattered light to the overall scattering intensity compared to non-activated platelets with no microparticles (FIG. 1, Sample 2).

Samples of platelet rich plasma (PRP) with high numbers of microparticles such as Sample 1 in FIG. 1 are considered to be activated.

The DLS test employs donor or patient platelets and detects their activation by measuring the changes induced by addition of patient serum and/or heparin. The platelet activation is measured by DLS in a special device optimized for testing microparticles in blood and blood products. Therapeutic levels of heparin added to the assay mix allow for the formation of PF4/heparin complexes which are recognized by pathologic HIT antibodies, form immune complexes (IC) and bind to the donor platelet surface. Binding of IC to sensitive donor platelets is expected to cause platelet activation and a significant, detectable change in the DLS size distribution profile. The specificity of the assay is increased by assessing the inhibition of platelet activation by (1) testing the effect of addition of a pathologic antibody to confirm a negative result and (2) by adding a high dose of heparin to confirm a positive result. Patient sample preparation follows the published SRA sample preparation steps¹.

### Procedure

Collect blood from normal donors, centrifuge and remove supernatant platelet-rich plasma (PRP) (TLX sample 1).

Alternatively, use an aliquot removed from a platelet concentrate.

Select sensitive donor platelets based on MP content; sensitive platelets may also be selected based on other DLS parameters obtained with ThromboLUX such as the TLX Score, the platelet radius or the polydispersity index.

Obtain patient PRP, plasma or preferably patient serum, collectively called the patient sample (TLX sample 2).

Mix donor platelets or platelet concentrate with patient serum and heparin.

Test immediately (t0), also referred to as the pre-reaction sample (TLX sample 3) and at least one additional time point, for example 4 additional times every 20 min after initial mixing (t20, t40, t60, t80) without changing the capillary.

The final test is referred to as the post-reaction samples ((TLX sample 4).

The difference in DLS size distributions between TLX sample 4 and TLX sample 3 is attributed to the reaction of platelets with heparin and the quantitation of the difference compared to a predetermined threshold is used to determine whether a reaction occurred or not.

### Confirmation of Results

If the test result is positive inhibit the reaction with excess heparin.

If the test result is negative add a pathological antibody.

Test immediately (t0) and at least one additional time point, for example 4 additional times every 20 min after initial mixing (t20, t40, t60, t80) without changing the capillary to evaluate the kinetics of the drug-induced changes on platelets

### Microparticle Test

This Standard Test Method is used to assay microparticles in samples prepared in the previous steps (TLX sample 1-4) using Dynamic Light Scattering. The assay is performed using the ThromboLUX instrument and system.

The ThromboLUX System performance has been verified using the compatible accessories supplied by the manufacturer. For purposes of the present invention, the ThromboLUX instrument is set up and operated according manufacturer's instructions.

### Data Analysis and Data Access

Following analysis, data are downloaded from the ThromboLUX instrument and software associated with the instrument (namely, software with the trademark ThromboSight and ThromboHIT) compares DLS parameters with previously determined thresholds to determine whether the sample is positive or negative for HIT. These results can be compared with results from other confirmatory tests and subjected to statistical analysis.

### Advantages of the Current Invention Over Previous Methods

The TLX-M test is practical and less resource intensive than current tests.

Sensitive donor platelets can be identified based on the % MP content of the PRP or platelet concentrate.

The size distribution results obtained with sensitive platelets and HIT-negative patient sera are significantly different from the size distribution results obtained with HIT-positive patient sera. The difference disappears when positive HIT sera are inhibited with a high dose of heparin or pathologic antibody is added to negative HIT sera.

The ThromboLUX-M microparticle (TLX-MP) test confirms HIT by two independent indicators:

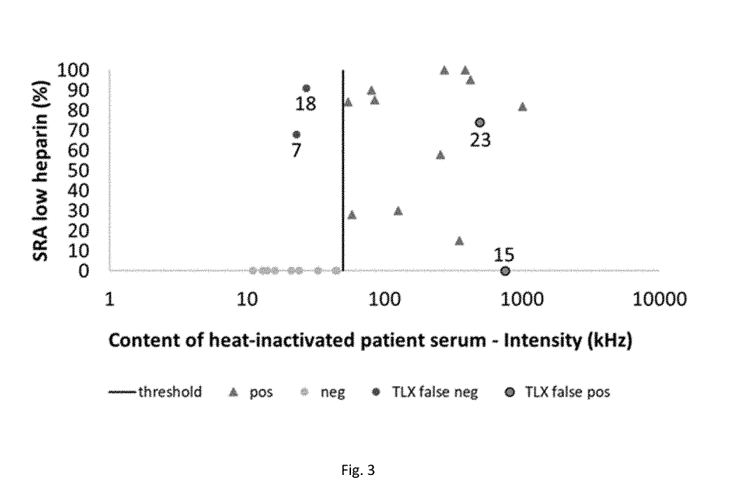

Composition of patient serum—obtained in a 5 min test following heat-inactivation that does not require fresh donor platelets (see FIG. 3).

Change in composition of the reaction mixture—donor platelets, heat-inactivated patient serum and heparin: Negative serum samples show no significant change while positive samples show significant changes in composition of the reaction mixture.

### Study

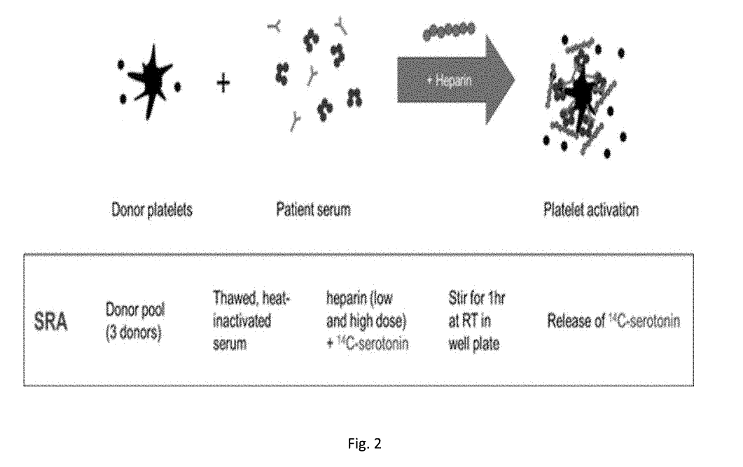

A study using patient serum samples with known SRA test results was conducted. FIG. 2, shows the overview of test sample preparation. The following conditions were tested: Donor platelet activation status (activated, non-activated, donor pool), thawed patient serum (naive or heat-inactivated), low-dose and high-dose heparin, and conditions to induce platelet activation with positive patient serum in the presence of heparin.

FIG. 2 illustrates an overview of test sample preparation for a confirmatory test for HIT. The box labeled SRA lists the conditions for the serotonin release assay. For the ThromboLUX-MP test the first indicator is the particle content of heat-inactivated serum alone (low content: negative, high content: positive); the second indicator is the change in sample composition (heparin+patient serum+donor platelet-rich plasma) during the reaction measured as a change in DLS size distributions between the pre- and post-reaction samples.

### Primary Objective

The primary objective of the study was to investigate if sample composition detected by dynamic light scattering (DLS) in mixtures of donor platelets with patient serum and heparin can confirm HIT. DLS results were compared to the results from current confirmatory assays (HIT antibody ELISA and serotonin release assay (SRA)). The hypothesis was that microparticle testing is equivalent to current confirmatory tests.

The study was started on the premise that the TLX-MP test measures the release of microparticles from donor platelets induced by the addition of patient serum in the presence of heparin. ThromboLUX is optimized for testing microparticles in blood and blood products which is why it was expected that it could be used to measure the content of patient sera as well as the reaction mixtures of those sera with donor platelets and heparin. The hypothesis was that therapeutic levels of heparin added to the mixture would allow the formation of PF4/heparin complexes which would form immune complexes (IC) with pathologic HIT antibodies that bind and activate donor platelets. It was further expected that donor platelet activation would cause a significant, detectable change in the DLS size distribution profile.

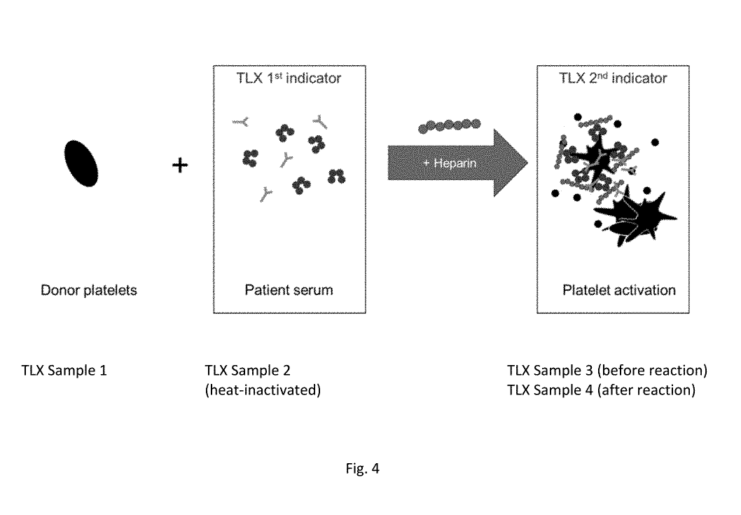

FIG. 4 shows a schematic overview of the confirmatory functional test using DLS. Note that TLX sample 3 in FIG. 4 was not tested but approximated by calculation from TLX samples 1 and 2. With respect to FIG. 4 it is notable that the methodology of the present invention evaluates the complete change in sample composition rather than just evaluating microparticles as a subgroup of particles—the assessment thus considers platelets, microparticles and aggregates.

### Test Development

The study assessment was conducted with left-over samples of patient sera and donor platelets. For each patient sample at least 3 ThromboLUX tests were performed and compared to current HIT confirmation tests, ELISA and SRA.

Table 3 lists the samples/conditions that were tested with ThromboLUX:

TABLE 3

| Sample | Condition | Finding |
|---|---|---|
| Donor platelet- | Single donor | The activation status of donor platelets was |
| rich plasma | Donor pool | determined and shown to contribute to the pool |
| (PRP) | | according to the volume; generally, about 60-70% |
| | | of donors have non-activated platelets with |
| | | no or low complement activation. |
| Patient serum | Naïve | Heat inactivation and centrifugation of patient |
| | Heat-inactivated | serum is required (rationale: inactivation of |
| | | complement and increase in content of small, |
| | | potentially reactive components). |
| Reaction | low dose heparin | Donor platelets react with patient serum and low |
| mixture | | dose heparin. |
| | Activated PRP | The activation status of donor platelets used in |
| | Non-activated PRP | the reaction mixture with heparin does not seem |
| | | to be critical for positive or negative samples; |
| | | however, low positive samples only react with |
| | | non-activated platelets. It is hypothesized that |
| | | activated PRP contains activated complement |
| | | factors and thus impacts the TLX-MP test (same |
| | | rationale as for heat-inactivating serum and |
| | | potential reason why some protocols for SRA |
| | | suggest washing platelets). |
| | Calculating DLS | The difference in size distributions, including |
| | signal of pre-reaction mixture | microparticle content, of the reaction mixture |
| | Measuring DLS | (heparin, patient serum, donor PRP) before and |
| | signal of pre-reaction mixture | after the reaction confirms the presence of |
| | | platelet-specific antibodies. |

The calculation of DLS may be used pre-reaction. The immediately tested pre-reaction mixture may in that instance be the post-reaction mixture since a reaction has occurred that has made the results from the actual mixture different from the calculated results.

During the study platelet pools and the platelet-rich plasma (PRP) of individual contributing donors were tested to determine how donors contributed to the pools. Platelet pools are used for the SRA to avoid the need of identifying pedigree donors. Thus, most reaction mixtures tested in the study used donor pools. Some samples were tested with activated and/or non-activated PRP from single donors.

Initially naïve and heat-inactivated patient serum was tested for one positive and one negative sample. Cleaner compositions and better reactions were observed with heat inactivated serum which was subsequently used for blinded samples.

### Rationale for Heat-Inactivation of Patient Sample

Heat-inactivation (heating to 56° C. for 30 minutes) is done to inactivate complement, a group of proteins present in sera that are part of the immune response. This is sometimes important for cells that will be used to prepare or assay viruses, or cells that are used in cytotoxicity assays or other systems where complement may have an unwanted influence. Heat-inactivation is also recommended for growing embryonic stem cells as well as for many insect cell lines. Heat has also been used to destroy mycoplasma in serum.

### Results/Findings

Negative serum samples had low particle content and showed no reaction with donor platelets and low dose heparin while positive samples had high particle content and showed significant changes in composition of the reaction mixture.

FIG. 3. Prediction of SRA-positivity by TLX-MP-measured content of heat-inactivated patient serum. From a ROC curve analysis (XLSTAT 2018.3, Addinsoft) a threshold was determined for maximum sensitivity and specificity. One false positive (sample 23) showed high serotonin release with low dose heparin but no inhibition with high dose heparin.

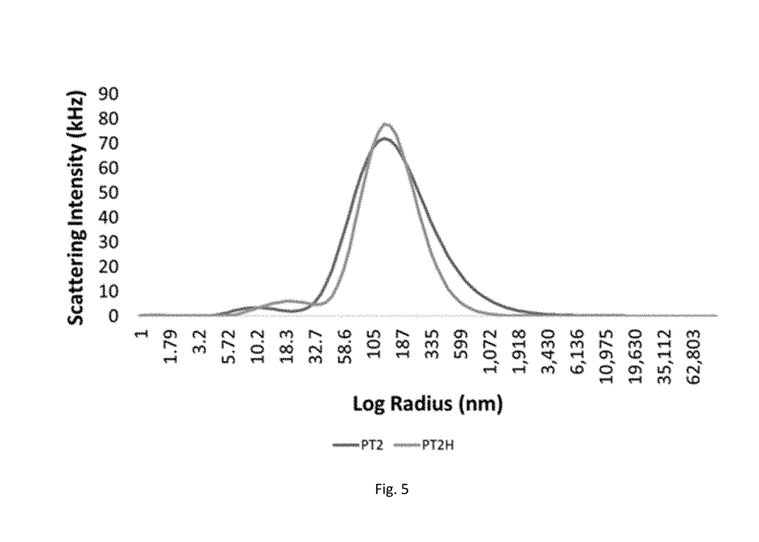

FIG. 5 illustrates the effect of heat-inactivation on patient serum. Large particles are removed resulting in a narrower distribution of microparticles with an average radius of 150 nm. Heat inactivation improves the DLS signal and result from thawed patient serum (TLX 1^{st} indicator).

With reference now to FIG. 6, calculation of reaction mixture signal is illustrated schematically. Change with reaction is the difference in size distributions between before and after the incubation and the particle content redistribution in the reaction mixture to obtain the reaction difference may be seen (i.e., the “reaction difference” curve of FIG. 6). Changes, also called residuals, are shown as the lowermost line in FIG. 6. The specific interaction of donor platelets with patient sample in the presence of heparin led to an increase in microparticles, a decrease in small platelets and an increase in aggregates as shown in the sift of the platelet peak to the right in the graph.

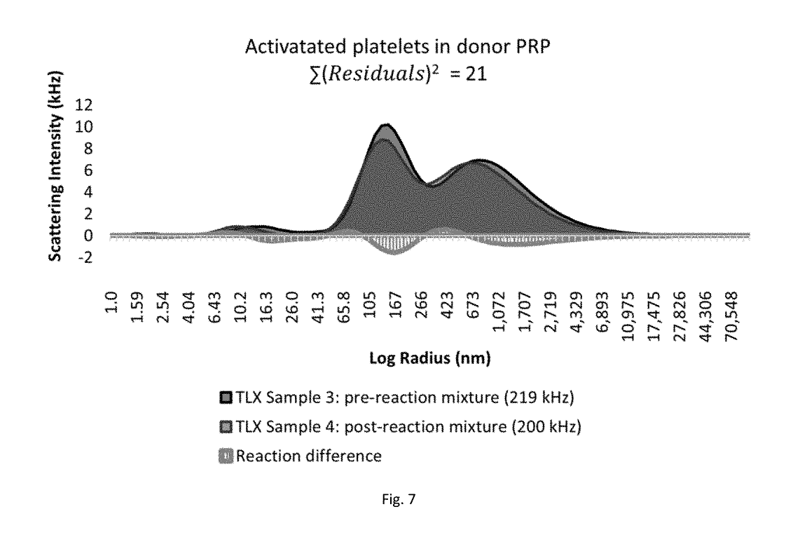

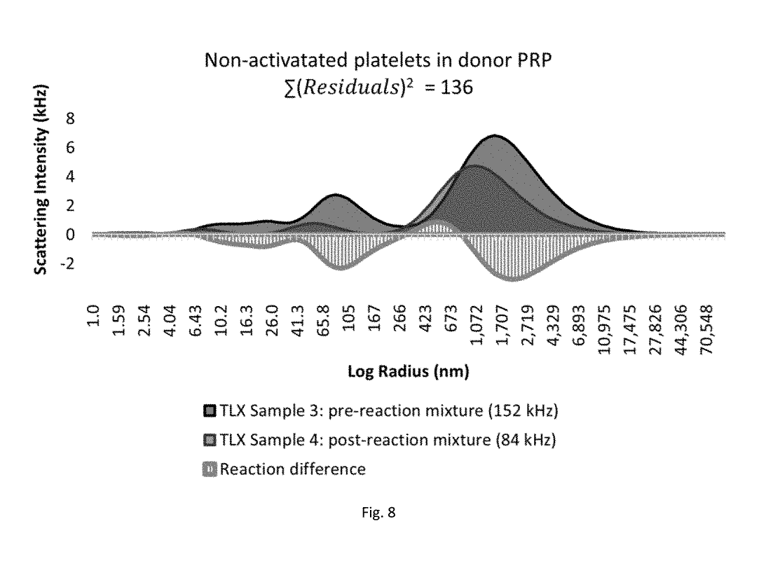

Mathematically, overall differences between curves are calculated as Σ(Residuals)² in FIGS. 7 and 8 in which the comparison of reaction change for a low positive sample with platelets from a fresh activated pool (Fig.7) and a fresh non-activated donor (FIG. 8) are shown. In FIG. 7 the overall difference is small because the assessment began with already-activated donor platelets, heparin, so the patient sample was only weakly positive. But as illustrated in FIG. 8, combining the weakly-positive patient sample with the non-activated donor platelets and heparin led to a significant decrease in almost all particle sizes due to the aggregation and settling out of the aggregates—illustrated by the significant drop of total intensity from 200 kHz of the reaction mixture in FIGS. 7 to 83 kHz of the reaction mixture in FIG. 8. The difference between the size distributions before and after the reaction with heparin shows that the reaction of non-activated platelets (FIG. 8) is more pronounced (TLX 2^{nd} indicator). The sum of the square of differences is a metric for overall differences widely used in regression analysis.

The use of non-activated donor PRP to identify low-positive HIT sera is preferred. No changes were seen in the absence of heparin.

1. The TLX-M test was shown to be practical and less resource intensive than current tests.

2. Donor platelets can be characterized based on the %MP content of the PRP and their contribution to the pool can be shown. The TLX-MP test seems to give better reaction results for low-positive samples when single donor non-activated platelets were used. Thus, it appeared that for the TLX-MP assay “sensitive platelets” correspond to non-activated platelets.

3. The size distribution results obtained with sensitive platelets and HIT-negative patient sera were significantly different from the size distribution results obtained with HIT-positive patient sera.

### Sensitivity and Specificity

The literature describes the “washed platelet SRA using optimal donors” as the gold standard diagnostic test for HIT with high sensitivity (>95%) and specificity for HIT²; the specificity of the SRA depends on the clinical situation, but in most circumstances is at least 95%. Previously, the highest level of accuracy was reached using a combination of ELISA with either SRA or platelet aggregation tests.³

### Conclusion

Two independent indicators for HIT are assessed with the TLX test:

1. TLX 1^{st} indicator: Patient serum composition (quick and requires no donor sample)

2. TLX 2^{nd} indicator: Change in sample composition with reaction: donor platelets+patient serum+heparin

TLX-MP test results from this study showed 85% sensitivity and 82% specificity for detecting SRA positive samples in less than 2 hrs.

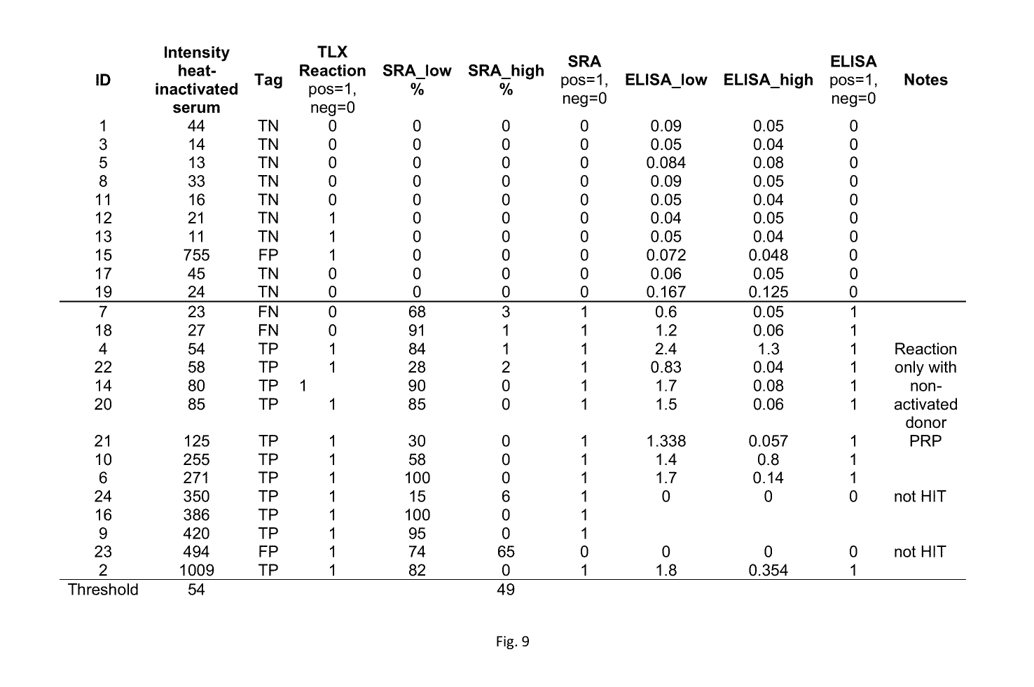

FIG. 9 is an overview of the results of the described study in tabular format.

The economic impact of the inventions described herein may be significant. The first application, the predictive test, is essentially a preventative prescreening for heparin sensitivity of a patient. If every patient receiving Heparin was prescreened at ˜100% sensitivity, the total financial savings of costs incurred from HIT would be somewhere in the range of $863 Million to $8.6 Billion annually for the total cost of HIT cases in the United States. The total cost incurred from the testing would be the cost of two tests for the 1^{st} and 2^{nd} indicator at $120 multiplied by the 12 000 000 patients who would receive Heparin. This $1.4 billion would be subtracted by the original savings to leave up to a $7.2 billion net gain from the testing annually. On the other end of the range $577 million could be lost due to the testing but lowering the risk to miss HIT. These financial costs do not include the 90,000 patients that die of HIT and related complications annually who could be saved by prescreening. These financial costs don't include the cost differences in anticoagulant treatment. According to Healthcare Blue Book a 30 day supply of enoxaparin (LMWH) costs $255 dollars. Warfarin is far cheaper at $12 per 30 days, yet when factoring in the cost of Warfarin checkups the total monthly costs are around the same. $255 for Heparin versus $188 -$244 for Warfarin. Fondaparinux is far more expensive at ˜$14,000 per month.

While the present invention has been described in terms of preferred and illustrated embodiments, it will be appreciated by those of ordinary skill that the spirit and scope of the invention is not limited to those embodiments, but extend to the various modifications and equivalents as defined in the appended claims.

1. A method of predicting the risk of drug-induced thrombocytopenia (DIT) comprising the steps of:
a. obtaining a first patient sample from a patient, the patient sample comprising platelets;
b. adding to the first patient sample a compound that is suspected to cause a reaction with platelets in the sample;
c. analyzing the patient sample to determine particle content;
d. based on the determined particle content, determining platelet activation in the sample as a measure of the risk of DIT.

2. The method according to claim 1 in which analysis of the sample to determine particle content is done with dynamic light scattering (DLS) and wherein the DLS analysis measures particle content in the sample for all particles contained therein.

3. The method according to claim 2 in which the particle content in the patient sample comprises platelets, microparticles and aggregates.

4. The method according to claim 2 including the step of comparing the determined platelet activation to predetermined thresholds.

5. The method according to claim 4 further comprising detecting a change in size distribution of particles in the sample.

6. The method according to claim 5 including detecting a change of relative content of particles smaller than 550 nm and a change in relative content of particles larger than 500 nm.

7. The method according to claim 2 in which platelet activation in the sample is determined by DLS determination of the mean particle radius.

8. The method according to claim 2 including using DLS to monitor changes in platelet activation over time.

9. The method according to claim 2 further confirming DIT according to the steps comprising:
a. obtaining a second patient sample from the patient;
b. obtaining a first sample of platelet rich plasma (PRP) from a pedigree donor;
c. adding to the first sample of PRP from the pedigree donor a compound that is suspected to cause a reaction with platelets in the PRP;
d. combining the second patient sample with the first sample of PRP from the pedigree donor;
e. analyzing the combination formed in step d. with DLS to determine platelet activation.

10. The method according to claim 9 in which the analysis in step e. is compared to a calculated DLS result combining the relative contributions of the first sample of PRP from the pedigree donor and the second patient sample from the patient.

11. The method according to claim 9 including the step of comparing the determined platelet activation to predetermined thresholds.

12. The method according to claim 9 in which the compound that is suspected to cause a reaction with platelets is heparin.

13. A method of detecting the assaying particles in a patient sample containing platelets in order to confirm drug-induced thrombocytopenia (DIT), the method comprising the steps of:
a. obtaining a first patient sample from a patient;
b. obtaining a second sample from a pedigree donor, the second sample comprising platelet rich plasma (PRP);
c. adding to the first patient sample a compound that is suspected to cause a reaction with the platelets;
d. combining the second sample with the first sample;
e. analyzing the combination formed in step d using dynamic light scattering (DLS) to determine platelet activation;
f. comparing the determined platelet activation to predetermined thresholds to determine DIT.

14. The method according to claim 13 wherein the DLS analysis comprises analysis of all particles in the combination.

15. The method according to claim 14 in which the combination comprises platelets, microparticles and aggregates.

16. The method according to claim 14 in which step e. further comprises detecting a change in size distribution of particles.

17. The method according to claim 16 including detecting a change of relative content of particles smaller than 550 nm and a change in relative content of particles larger than 500 nm.

18. A method of assessing drug-induced thrombocytopenia (DIT) in a patient sample containing platelets, the method comprising the steps of:
a. obtaining a first patient sample;
b. adding to the first patient sample a compound that is suspected to cause a reaction with platelets in the sample;
c. analyzing the first patient sample using dynamic light scattering (DLS) to determine particle content;
d. based on the determined particle content, determining platelet activation in the first patient sample and comparing the platelet activation to a predetermined threshold to thereby determine if DIT is an indicated risk;
e. if DIT is an indicated risk in step d.:
i. obtaining a second patient sample;
ii obtaining a first sample of platelet rich plasma (PRP) from a pedigree donor;
iii. adding to the first sample of PRP from the pedigree donor a compound that is suspected to cause a reaction with platelets in the PRP;
iv. combining the second patient sample with the first sample of PRP from the pedigree donor;
v. analyzing the combination formed in step e.iv. with DLS to determine platelet activation to thereby confirm that the compound that is suspected to cause a reaction with platelets in the sample may cause DIT in the patient.

19. The method according to claim 18 in which the DLS analysis in steps c. and e.v. comprises detecting a change in size distribution of all particles in the sample.

20. The method according to claim 19 including detecting a change of relative content of particles smaller than 550 nm and a change in relative content of particles larger than 500 nm.
